SEQUENCE LISTING (15 sequences)

SEQ ID NO: 1 - protein, 127 aa, Artificial Sequence
  Synthetic
EVQLVESGGGLVQPGRSLRLSCVASGFTFNDYAMHWVRQAPGKGLEWVSVISWNSDSIGYADSVKGRFTISRDNAKNSLYLQMHSLRAEDTALYYCAKDNHYGSGSYYYYQYGMDVWGQGTTVTVSS

SEQ ID NO: 2 - protein, 108 aa, Artificial Sequence
  Synthetic
EIVMTQSPATLSVSPGERATLSCRASQSVSSNLAWYQQKPGQAPRLLIYGASTRATGIPARFSGSGSGTEFTLTISSLQSEDFAVYYCQHYINWPLTFGGGTKVEIKR

SEQ ID NO: 3 - protein, 123 aa, Artificial Sequence
  Synthetic
EVQLVESGGGLVQPGRSLRLSCAASGFTFDDYTMHWVRQAPGKGLEWVSGISWNSGSIGYADSVKGRFTISRDNAKKSLYLQMNSLRAEDTALYYCAKDNSGYGHYYYGMDVWGQGTTVTVAS

SEQ ID NO: 4 - protein, 8 aa, Artificial Sequence
  Synthetic
GFTFNDYA

SEQ ID NO: 5 - protein, 8 aa, Artificial Sequence
  Synthetic
ISWNSDSI

SEQ ID NO: 6 - protein, 20 aa, Artificial Sequence
  Synthetic
AKDNHYGSGSYYYYQYGMDV

SEQ ID NO: 7 - protein, 6 aa, Artificial Sequence
  Synthetic
QSVSSN

SEQ ID NO: 8 - protein, 3 aa, Artificial Sequence
  Synthetic
GAS

SEQ ID NO: 9 - protein, 9 aa, Artificial Sequence
  Synthetic
QHYINWPLT

SEQ ID NO: 10 - protein, 8 aa, Artificial Sequence
  Synthetic
GFTFDDYT

SEQ ID NO: 11 - protein, 8 aa, Artificial Sequence
  Synthetic
ISWNSGSI

SEQ ID NO: 12 - protein, 16 aa, Artificial Sequence
  Synthetic
AKDNSGYGHYYYGMDV

SEQ ID NO: 13 - protein, 326 aa, Artificial Sequence
  Synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSLGK

SEQ ID NO: 14 - protein, 326 aa, Artificial Sequence
  Synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 15 - protein, 108 aa, Artificial Sequence
  Synthetic
AEIVMTQSPATLSVSPGERATLSCRASQSVSSNLAWYQQKPGQAPRLLIYGASTRATGIPARFSGSGSGTEFTLTISSLQSEDFAVYYCQHYINWPLTFGGGTKVEIK

(19) United States
(12) Patent Application Publication
(10) Pub. No.: US 2017/0174781 A1
(43) Pub. Date: Jun. 22, 2017

(54) Bispecific Anti-CD20/Anti-CD3 Antibodies to Treat Acute Lymphoblastic Leukemia

(71) Applicant: Regeneron Pharmaceuticals, Inc., Tarrytown, NY (US)

(72) Inventor: Carrie Brownstein, New York, NY (US)

(21) Appl. No.: 15/386,443

(22) Filed: Dec. 21, 2016

Related U.S. Application Data
(60) Provisional application No. 62/306,031, filed on Mar. 9, 2016.
(60) Provisional application No. 62/270,749, filed on Dec. 22, 2015.

(51) Int. Cl. C07K 16/30; C07K 19/00; A61K 45/06; A61K 39/395; C07K 16/28; C07K 16/46

(52) U.S. Cl. CPC C07K 16/30; C07K 16/2809; C07K 16/468; A61K 45/06; A61K 39/39558; C07K 16/3061; C07K 19/00; C07K 2317/31; C07K 2317/565; C07K 2317/56; C07K 2317/52; C07K 2317/53; A61K 2039/505

(57) ABSTRACT

The present invention provides methods for treating, reducing the severity, or inhibiting the growth of acute lymphoblastic leukemia. The methods of the present invention comprise administering to a subject in need thereof a therapeutically effective amount of a bispecific antibody that specifically binds to CD20 and CD3.

### CROSS-REFERENCE TO RELATED APPLICATIONS

This application claims the benefit under 35 USC §119(e) of U.S. provisional application No. 62/306,031, filed Mar. 9, 2016, and U.S. provisional application No. 62/270,749, filed Dec. 22, 2015, each of which is herein specifically incorporated by reference in its entirety.

### REFERENCE TO A SEQUENCE LISTING

This application incorporates by reference the Sequence Listing submitted in computer readable form as file 10241US01-Sequence.txt, created on Dec. 19, 2016, and containing 12,179 bytes.

### FIELD OF THE INVENTION

The present invention resides in the field of medicine, and relates to methods for treating acute lymphoblastic leukemia (ALL) via administration of a therapeutically effective amount of an antibody that specifically binds to CD20 and CD3 to a subject in need thereof.

### BACKGROUND

B-cell cancers are a group of heterogeneous cancers of the white blood cells known as B-lymphocytes and include leukemias (located in the blood) and lymphomas (located in the lymph nodes). Acute lymphoblastic leukemia (ALL) is a malignant (clonal) disease of the bone marrow in which early lymphoid precursors proliferate and replace the normal hematopoietic cells of the marrow (Seiter, K. “Acute Lymphoblastic Leukemia”. Medscape Reference. WebMD. Retrieved Mar. 6, 2016). ALL is an aggressive malignancy, characterized by a sudden onset and rapid progression. ALL may be classified by cell lineage (B cell or T cell), cell type (precursor or mature) and presence or absence of the Philadelphia (Ph) chromosome translocation. Relapsed or refractory (r/r) acute lymphoblastic leukemia (ALL) in adults has a poor prognosis when treated with conventional therapy. Only 7-12% of these patients become long-term survivors. (Saltman, D. et al., 2015, BMC Cancer 15:771; Fielding A. K. et al., Blood 2007; 109:944-950, Faderl S., et al., Cancer 2010; 116:1165-1176).) Novel approaches are urgently needed to improve the outcomes for this patient population.

Most B-cell cancers express CD20 on the cell surface of mature B cells. Methods for treating cancer by targeting CD20 are known in the art. For example, the chimeric anti-CD20 monoclonal antibody rituximab has been used or suggested for use in treating cancers such as NHL, chronic lymphocytic leukemia (CLL) and small lymphocytic lymphoma (SLL), either as monotherapy but more typically in combination with chemotherapy. Although anti-CD20 tumor targeting strategies have shown great promise in clinical settings, not all patients respond to anti-CD20 therapy, and some patients have been shown to develop resistance to or exhibit incomplete responses to anti-CD20 therapy (e.g., partial depletion of peripheral B-cells), for reasons that are not well understood (but which typically do not include loss of CD20 expression). Some patients relapse with a more aggressive phenotype or chemotherapy-resistant disease. Many patients with aggressive lymphomas have poor prognosis and less than 50% chance of relapse-free survival.

The prognosis for patients who relapse or are refractory to therapy remains dismal with median survival after salvage therapy of 2 to 8 months. In addition, high-dose chemotherapy leads to severe adverse side effects. Thus, there is a high unmet need for therapies that are effective, prevent relapse and have less side effects for patients with B-cell cancers.

CD3 is a homodimeric or heterodimeric antigen expressed on T cells in association with the T cell receptor complex (TCR) and is required for T cell activation. Antibodies against CD3 have been shown to cluster CD3 on T cells, thereby causing T cell activation in a manner similar to the engagement of the TCR by peptide-loaded MHC molecules. Bispecific monoclonal antibodies designed to target both CD20 and CD3 bridge CD20-expressing cells with cytotoxic T cells, result in CD20-directed polyclonal T cell killing.

### BRIEF SUMMARY OF THE INVENTION

According to certain embodiments, the present invention provides methods for treating, ameliorating at least one symptom or indication, or inhibiting the growth or progression of acute lymphoblastic leukemia in a subject. The methods according to this aspect of the invention comprise administering a therapeutically effective amount of an antibody or antigen-binding fragment thereof that specifically binds to CD20 and CD3 to a subject in need thereof.

In certain embodiments of the present invention, methods are provided for treating, ameliorating at least one symptom or indication, or inhibiting the growth of cancer in a subject. In certain embodiments of the present invention, methods are provided for delaying the growth of leukemic cells or preventing leukemic cell recurrence. The methods, according to this aspect of the invention, comprise administering one or more doses of a therapeutically effective amount of a bispecific antibody that specifically binds to CD20 and CD3 to a subject in need thereof.

In certain embodiments, the cancer is acute lymphoblastic leukemia. In certain embodiments, each dose of the bispecific antibody against CD20 and CD3 comprises 0.1-10 mg/kg of the subject's body weight. In certain embodiments, each dose of the bispecific antibody against CD20 and CD3 comprises 4 mg/kg of the subject's body weight. In certain embodiments, each dose of the bispecific antibody comprises 10-5000 micrograms.

In some cases, the bispecific antibody is administered intravenously, subcutaneously, or intraperitoneally.

In certain embodiments, the methods of the present invention comprise administering 0-50 therapeutic doses of a bispecific antibody against CD20 and CD3, wherein each dose is administered 0.5-12 weeks after the immediately preceding dose.

In certain embodiments, the subject is resistant or inadequately responsive to, or relapsed after, prior therapy. In some cases, the treatment produces a therapeutic effect selected from the group consisting of delay in reduction in leukemic cell number, increase in survival, partial response, and complete response. In some embodiments, the therapeutic effect is an increase in survival as compared to an untreated subject. In some embodiments, the leukemic cell number is reduced by at least 50% as compared to an untreated subject.

In certain embodiments, the bispecific antibody is administered in combination with a second therapeutic agent or therapy. In certain cases, the second therapeutic agent or therapy is selected from the group consisting of radiation, surgery, a chemotherapeutic agent, a cancer vaccine, a PD-1 inhibitor, a PD-L1 inhibitor, a LAG-3 inhibitor, a CTLA-4 inhibitor, a TIM3 inhibitor, a BTLA inhibitor, a TIGIT inhibitor, a CD47 inhibitor, an indoleamine-2,3-dioxygenase (IDO) inhibitor, a vascular endothelial growth factor (VEGF) antagonist, an angiopoietin-2 (Ang2) inhibitor, a transforming growth factor beta (TGFβ) inhibitor, an epidermal growth factor receptor (EGFR) inhibitor, an antibody to a tumor-specific antigen, Bacillus Calmette-Guerin vaccine, granulocyte-macrophage colony-stimulating factor, a cytotoxin, an interleukin 6 receptor (IL-6R) inhibitor, an interleukin 4 receptor (IL-4R) inhibitor, an IL-10 inhibitor, IL-2, IL-7, IL-21, IL-15, an antibody-drug conjugate, an anti-inflammatory drug, and a dietary supplement.

In a preferred embodiment, the bispecific antibody that binds to CD20 and CD3 comprises: (i) a first antigen-binding arm comprising the heavy chain CDRs (A-HCDR1, A-HCDR2 and A-HCDR3) of a HCVR (A-HCVR) of SEQ ID NO: 1 and the light chain CDRs (LCDR1, LCDR2 and LCDR3) of a LCVR of SEQ ID NO: 2; and (ii) a second antigen-binding arm comprising the heavy chain CDRs (B-HCDR1, B-HCDR2 and B-HCDR3) of a HCVR (B-HCVR) of SEQ ID NO: 3 and the light chain CDRs (LCDR1, LCDR2 and LCDR3) of a LCVR of SEQ ID NO: 2.

According to certain embodiments, A-HCDR1 comprises the amino acid sequence of SEQ ID NO: 4; A-HCDR2 comprises the amino acid sequence of SEQ ID NO: 5; A-HCDR3 comprises the amino acid sequence of SEQ ID NO: 6; LCDR1 comprises the amino acid sequence of SEQ ID NO: 7; LCDR2 comprises the amino acid sequence of SEQ ID NO: 8; and LCDR3 comprises the amino acid sequence of SEQ ID NO: 9. According to certain embodiments, the A-HCVR comprises the amino acid sequence of SEQ ID NO: 1 and the LCVR comprises the amino acid sequence of SEQ ID NO: 2.

According to certain embodiments, the second antigen-binding arm of the bispecific antibody comprises three heavy chain CDRs (B-HCDR1, B-HCDR2 and B-HCDR3) of a heavy chain variable region (B-HCVR) comprising the amino acid sequence of SEQ ID NO: 3 and three light chain CDRs (LCDR1, LCDR2 and LCDR3) of a light chain variable region (LCVR) comprising the amino acid sequence of SEQ ID NO: 2. In some cases, B-HCDR1 comprises the amino acid sequence of SEQ ID NO: 10; B-HCDR2 comprises the amino acid sequence of SEQ ID NO: 11; B-HCDR3 comprises the amino acid sequence of SEQ ID NO: 12; LCDR1 comprises the amino acid sequence of SEQ ID NO: 7; LCDR2 comprises the amino acid sequence of SEQ ID NO: 8; and LCDR3 comprises the amino acid sequence of SEQ ID NO: 9. In some embodiments, the B-HCVR comprises the amino acid sequence of SEQ ID NO: 3 and the LCVR comprises the amino acid sequence of SEQ ID NO: 2.

In any of the embodiments of the anti-CD20/anti-CD3 antibody discussed above or herein, the LCVR can alternatively comprise the amino acid sequence of SEQ ID NO:15.

In a preferred embodiment, the bispecific antibody is REGN1979.

In certain embodiments, the subject has CD20 expression on ≧0% of leukemic lymphoblasts, as determined by flow cytometry. In some cases, the subject has CD20 expression on 5% of leukemic lymphoblasts, as determined by flow cytometry. In some cases, the subject has CD20 expression on ≧20% of leukemic lymphoblasts, as determined by flow cytometry.

In certain embodiments, the bispecific antibody comprises a chimeric Fc domain tethered to each of the first and second antigen-binding domains. In certain embodiments, the chimeric Fc domain comprises a chimeric hinge.

In another aspect, the present invention provides use of a bispecific antibody against CD20 and CD3 in the manufacture of a medicament to treat or inhibit the growth of cancer in a subject, including humans. In certain embodiments, the cancer is a B-cell cancer. In a preferred embodiment, the cancer is acute lymphoblastic leukemia.

Other embodiments of the present invention will become apparent from a review of the ensuing detailed description.

### BRIEF DESCRIPTION OF THE FIGURES

FIG. 1 shows a tumor volume (in mm³) study in NSG mice implanted subcutaneously with a mixture of Raji tumor cells and PBMCs in which a CD3×CD20 bispecific antibody of the invention (Ab 1, also known as REGN1979) at 0.4 mg/kg, 2×/week (i.p), irrelevant antibody Control Ab 6 at 0.4 mg/kg, 2×/week (i.p), or vehicle was compared to rituximab, anti-CD20 antibody at 8 mg/kg, 5×/week (i.p), and CD19×CD3 BiTE at 0.5 mg/kg, 5×/week (i.v). (For CD19×CD3 BiTE, see Nagorsen D, et al. Pharmacol Ther. 2012 December; 136(3):334-42, 2012.) Treatment was administered to mice with established tumors (˜100-500 mm3). Data are expressed as mean (SEM) and were subjected to ANOVA analysis. Ab1, which was dosed 2× per week i.p., was comparable to the potency of CD19×CD3 BiTE which was dosed 5×/week i.v. in this in vivo model.

FIG. 2 shows a tumor volume (in mm³) study in NSG mice implanted subcutaneously with Raji/PBMC mixture, analogously to FIG. 1, with ANOVA analysis provided for Ab 1 (also known as REGN1979), Control Ab 6, rituximab and vehicle control. Ab 1 (also known as REGN1979) dosed 2× per week was superior to rituximab therapy (dosed at 8 mg/kg; 5×/week i.p.) in suppressing established Raji tumors.

FIG. 3 depicts a dosing regimen for an anti-CD20/anti-CD3 bispecific antibody in a clinical trial for ALL.

### DETAILED DESCRIPTION

Before the present invention is described, it is to be understood that this invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice of the present invention, the preferred methods and materials are now described. All publications mentioned herein are incorporated herein by reference to describe in their entirety.

### Methods for Treating ALL or Inhibiting the Progression of ALL

The present invention includes methods for treating, ameliorating or reducing the severity of at least one symptom or indication, or inhibiting the progression of acute lymphoblastic leukemia in a subject. The methods according to this aspect of the invention comprise administering a therapeutically effective amount of a bispecific antibody against CD20 and CD3 to a subject in need thereof. As used herein, the terms “treat”, “treating”, or the like, mean to alleviate symptoms, eliminate the causation of symptoms either on a temporary or permanent basis, to delay or inhibit tumor cell growth, to reduce tumor cell load or tumor burden, to promote tumor regression, to cause tumor shrinkage, necrosis and/or disappearance, to prevent tumor recurrence, and/or to increase duration of survival of the subject.

As used herein, the expression “a subject in need thereof” means a human or non-human mammal that exhibits one or more symptoms or indications of cancer, and/or who has been diagnosed with ALL and who needs treatment for the same. In many embodiments, the term “subject” may be interchangeably used with the term “patient”. For example, a human subject may be diagnosed with a primary or a metastatic tumor and/or with one or more symptoms or indications including, but not limited to, enlarged lymph node(s), swollen abdomen, chest pain/pressure, unexplained weight loss, fever, night sweats, persistent fatigue, loss of appetite, enlargement of spleen, itching. In specific embodiments, the expression includes human subjects that have and need treatment for acute lymphoblastic leukemia (ALL). In other specific embodiments, the expression includes subjects with CD20+ B-lineage ALL (e.g., defined as CD20 expression as determined by flow cytometry on ≧20% of leukemic lymphoblasts).

In a preferred embodiment, “a subject in need thereof” refers to subjects with CD20+ B-lineage ALL defined as CD20 expression as determined by flow cytometry on ≧5% of leukemic lymphoblasts. In a more preferred embodiment, the expression refers to subjects with CD20+ B-lineage ALL defined as CD20 expression as determined by flow cytometry on ≧0% of leukemic lymphoblasts.

In an embodiment, the expression includes subjects with CD20 expression as determined by flow cytometry on ≧9% of leukemic lymphoblasts. In an embodiment, the expression includes subjects with CD20 expression as determined by flow cytometry on ≧8% of leukemic lymphoblasts. In an embodiment, the expression includes subjects with CD20 expression as determined by flow cytometry on ≧7% of leukemic lymphoblasts. In an embodiment, the expression includes subjects with CD20 expression as determined by flow cytometry on ≧6% of leukemic lymphoblasts. In an embodiment, the expression includes subjects with CD20 expression as determined by flow cytometry on ≧5% of leukemic lymphoblasts. In an embodiment, the expression includes subjects with CD20 expression as determined by flow cytometry on ≧14% of leukemic lymphoblasts. In an embodiment, the expression includes subjects with CD20 expression as determined by flow cytometry on ≧3% of leukemic lymphoblasts. In an embodiment, the expression includes subjects with CD20 expression as determined by flow cytometry on ≧2% of leukemic lymphoblasts. In an embodiment, the expression includes subjects with CD20 expression as determined by flow cytometry on ≧11% of leukemic lymphoblasts. In an embodiment, the expression includes subjects with CD20 expression as determined by flow cytometry on ≧0% of leukemic lymphoblasts. In an embodiment, the expression includes subjects with CD20 expression as determined by flow cytometry on ≧9% of leukemic lymphoblasts. In an embodiment, the expression includes subjects with CD20 expression as determined by flow cytometry on ≧8% of leukemic lymphoblasts. In an embodiment, the expression includes subjects with CD20 expression as determined by flow cytometry on ≧7% of leukemic lymphoblasts. In an embodiment, the expression includes subjects with CD20 expression as determined by flow cytometry on ≧6% of leukemic lymphoblasts. In an embodiment, the expression includes subjects with CD20 expression as determined by flow cytometry on ≧5% of leukemic lymphoblasts. In an embodiment, the expression includes subjects with CD20 expression as determined by flow cytometry on ≧4% of leukemic lymphoblasts. In an embodiment, the expression includes subjects with CD20 expression as determined by flow cytometry on ≧3% of leukemic lymphoblasts. In an embodiment, the expression includes subjects with CD20 expression as determined by flow cytometry on ≧2% of leukemic lymphoblasts. In an embodiment, the expression includes subjects with CD20 expression as determined by flow cytometry on ≧1% of leukemic lymphoblasts.

In certain embodiments, the expression “a subject in need thereof” includes patients with ALL that is relapsed or refractory to or is inadequately controlled by prior therapy (e.g., treatment with a conventional anti-cancer agent). For example, the expression includes subjects who have been treated with rituximab, Blinatumomab, JCAR014/JCAR015, CTL019, KTE-C19, Inotuzumab Ozogamicin (10), ⁹⁰Y-Epratuzumab-tetraxetan, chemotherapy, or an immune-modulating agent such as a blocker of CTLA, IBB, LAGS or OX-40. The expression also includes subjects with ALL for which conventional anti-cancer therapy is inadvisable, for example, due to toxic side effects. For example, the expression includes patients who have received one or more cycles of chemotherapy with toxic side effects. In certain embodiments, the expression “a subject in need thereof” includes patients with ALL which has been treated but which has subsequently relapsed or metastasized. For example, patients with ALL that may have received treatment with one or more anti-cancer agents leading to tumor regression; however, subsequently have relapsed with cancer resistant to the one or more anti-cancer agents (e.g., chemotherapy-resistant cancer) are treated with the methods of the present invention. In certain embodiments, the expression “a subject in need thereof” includes adults or children diagnosed with ALL having at least 1% CD20+ B-cell lineage leukemic lymphoblasts. In certain embodiments, the the expression “a subject in need thereof” includes adults or children diagnosed with ALL having at least 1% CD20+ B-cell lineage leukemic lymphoblasts and having ALL that is relapsed or refractory to or is inadequately controlled by prior therapy (e.g., treatment with a conventional anti-cancer agent).

The expression “a subject in need thereof” also includes subjects who are at risk of developing a ALL, e.g., persons with a family history of ALL, or persons with an immune system compromised due to HIV infection or due to immunosuppressive medications.

In certain embodiments, the methods of the present invention may be used to treat patients that show elevated levels of one or more cancer-associated biomarkers (e.g., CD20). For example, the methods of the present invention comprise administering a bispecific anti-CD20/anti-CD3 antibody to a patient with an elevated level CD20. In certain embodiments, the methods of the present invention are used in a subject with ALL. The terms “tumor”, “cancer” and “malignancy” are interchangeably used herein. The term “B-cell cancer”, as used herein, refers to tumors of white blood cells known as B-lymphocytes and includes leukemias (located in the blood) and lymphomas (located in the lymph nodes). The present invention includes methods to treat acute lymphoblastic leukemia. In certain embodiments, B-cell cancer includes, but is not limited to, acute lymphoblastic leukemia.

According to certain embodiments, the present invention includes methods for treating, or delaying or inhibiting the growth of a tumor. In certain embodiments, the present invention includes methods to promote tumor regression. In certain embodiments, the present invention includes methods to reduce tumor cell load or to reduce tumor burden. In certain embodiments, the present invention includes methods to prevent tumor cell recurrence. The methods, according to this aspect of the invention, comprise sequentially administering a therapeutically effective amount of a bispecific anti-CD20/anti-CD3 antibody to a subject in need thereof, wherein the antibody is administered to the subject in multiple doses, e.g., as part of a specific therapeutic dosing regimen. For example, the therapeutic dosing regimen may comprise administering one or more doses of a therapeutically effective amount of a bispecific anti-CD20/anti-CD3 antibody, wherein the one or more doses of the bispecific antibody are administered to the subject at a frequency of about once a day, once every two days, once every three days, once every four days, once every five days, once every six days, once a week, once every two weeks, once every three weeks, once every four weeks, once a month, once every two months, once every three months, once every four months, or less frequently. In certain embodiments, a dose of the bispecific antibody can be split into two or more fractions for separate administration within a given dosing period. Such fractional or split dosing can be used to reduce or eliminate the production of cytokines in response to the administration of the bispecific antibody, which is often referred to as a “cytokine storm” or “cytokine release syndrome.” In certain embodiments, each dose is split into from two to five fractions for administration within the dosing period. For example, a 1000 microgram (mcg) dose to be administered weekly can be divided into two 500 mcg doses for administration at different times within the one week dosing schedule. In certain embodiments, each dose can be split into from two to four fractions, or two or three fractions. In certain embodiments, each dose is split into 2 fractions. In certain embodiments, each does is split into 3 fractions. In certain embodiments, each does is split into 4 fractions. In certain embodiments, each does is split into 5 fractions. Such fractional dosing can be applied to, e.g., the doses discussed in paragraphs 0088-0090, below. In certain embodiments, a dose of the bispecific antibody is split into 2 or more fractions, wherein each fraction comprises an amount of the antibody equal to the other fractions. For example, a dose of anti-CD20/anti-CD3 antibody comprising 1000 micrograms may be administered once a week, wherein the dose is administered in 2 fractions within the week, each fraction comprising 500 micrograms. In certain embodiments, a dose of the bispecific antibody is administered split into 2 or more fractions, wherein the fractions comprise unequal amounts of the antibody, e.g., more than or less than the first fraction. For example, a dose of anti-CD20/anti-CD3 antibody comprising 1000 micrograms may be administered once a week, wherein the dose is administered in 2 fractions within the week, wherein the first fraction comprises 700 micrograms and the second fraction comprises 300 micrograms. As another example, a dose of anti-CD20/anti-CD3 antibody comprising 1000 micrograms may be administered once in 2 weeks, wherein the dose is administered in 3 fractions within the 2-week period, wherein the first fraction comprises 400 micrograms, the second fraction comprises 300 micrograms and the third fraction comprises 300 micrograms.

In certain embodiments, the present invention includes methods to inhibit, retard or stop tumor metastasis or tumor infiltration into peripheral organs. The methods, according to this aspect, comprise administering a therapeutically effective amount of a bispecific anti-CD20/anti-CD3 antibody.

In specific embodiments, the present invention provides methods for increased anti-tumor efficacy or increased tumor inhibition. The methods, according to this aspect of the invention, comprise administering to a subject with ALL a therapeutically effective amount of a bispecific anti-CD20/anti-CD3 antibody.

In certain embodiments, the methods of the present invention comprise administering a therapeutically effective amount of a bispecific anti-CD20/anti-CD3 antibody to a subject with ALL. In certain embodiments, the subject is not responsive to prior therapy or has relapsed after prior therapy.

In certain embodiments, the methods of the present invention comprise administering a therapeutically effective amount of a bispecific anti-CD20/anti-CD3 antibody to a subject with ALL. In certain embodiments, the subject is not responsive to prior therapy or has relapsed after prior therapy (e.g., with an anti-CD20 agent such as rituximab, or with Blinatumomab, JCAR014/JCAR015, CTL019, KTE-C19, Inotuzumab Ozogamicin (10), or ⁹⁰Y-Epratuzumab-tetraxetan).

In certain embodiments, the methods of the present invention comprise administering a bispecific anti-CD20/anti-CD3 antibody to a subject in need thereof as a “first line” treatment (e.g., initial treatment). In other embodiments, a bispecific anti-CD20/anti-CD3 antibody is administered as a “second line” treatment (e.g., after prior therapy). For example, a bispecific anti-CD20/anti-CD3 antibody is administered as a “second line” treatment to a subject that has relapsed after prior therapy with, e.g., chemotherapy, rituximab, Blinatumomab, JCAR014/JCAR015, CTL019, KTE-C19, Inotuzumab Ozogamicin (10), or ⁹⁰Y-Epratuzumab-tetraxetan.

In certain embodiments, the methods of the present invention are used to treat a patient with a MRD-positive disease. Minimum residual disease (MRD) refers to small numbers of cancer cells that remain in the patient during or after treatment, wherein the patient may or may not show symptoms or signs of the disease. Such residual cancer cells, if not eliminated, frequently lead to relapse of the disease. The present invention includes methods to inhibit and/or eliminate residual cancer cells in a patient upon MRD testing. MRD may be assayed according to methods known in the art (e.g., MRD flow cytometry). The methods, according to this aspect of the invention, comprise administering a bispecific anti-CD20/anti-CD3 antibody to a subject in need thereof.

The methods of the present invention, according to certain embodiments, comprise administering to a subject a therapeutically effective amount of a bispecific anti-CD20/anti-CD3 antibody in combination with a second therapeutic agent. The second therapeutic agent may be an agent selected from the group consisting of, e.g., radiation, chemotherapy, surgery, a cancer vaccine, a PD-L1 inhibitor (e.g., an anti-PD-L1 antibody), a LAG3 inhibitor (e.g., an anti-LAG3 antibody), a CTLA-4 inhibitor, a TIM3 inhibitor, a BTLA inhibitor, a TIGIT inhibitor, a CD47 inhibitor, an indoleamine-2,3-dioxygenase (IDO) inhibitor, a vascular endothelial growth factor (VEGF) antagonist, an Ang2 inhibitor, a transforming growth factor beta (TGFβ) inhibitor, an epidermal growth factor receptor (EGFR) inhibitor, an antibody to a tumor-specific antigen (e.g., CA9, CA125, melanoma-associated antigen 3 (MAGES), carcinoembryonic antigen (CEA), vimentin, tumor-M2-PK, prostate-specific antigen (PSA), mucin-1, MART-1, and CA19-9), a vaccine (e.g., Bacillus Calmette-Guerin), granulocyte-macrophage colony-stimulating factor, a cytotoxin, a chemotherapeutic agent, an IL-6R inhibitor, an IL-4R inhibitor, an IL-10 inhibitor, a cytokine such as IL-2, IL-7, IL-21, and IL-15, an anti-inflammatory drug such as corticosteroids, and non-steroidal anti-inflammatory drugs, and a dietary supplement such as anti-oxidants. In certain embodiments, the antibodies may be administered in combination with therapy including a chemotherapeutic agent, radiation and surgery. As used herein, the phrase ‘in combination with” means that the bispecific anti-CD20/anti-CD3 antibody is administered to the subject at the same time as, just before, or just after administration of the second therapeutic agent. In certain embodiments, the second therapeutic agent is administered as a co-formulation with the bispecific anti-CD20/anti-CD3 antibody. In a related embodiment, the present invention includes methods comprising administering a therapeutically effective amount of a bispecific anti-CD20/anti-CD3 antibody to a subject who is on a background anti-cancer therapeutic regimen. The background anti-cancer therapeutic regimen may comprise a course of administration of, e.g., a chemotherapeutic agent, or radiation. The bispecific anti-CD20/anti-CD3 antibody may be added on top of the background anti-cancer therapeutic regimen. In some embodiments, the bispecific anti-CD20/anti-CD3 antibody is added as part of a “background step-down” scheme, wherein the background anti-cancer therapy is gradually withdrawn from the subject over time (e.g., in a stepwise fashion) while the bispecific anti-CD20/anti-CD3 antibody is administered to the subject at a constant dose, or at an increasing dose, or at a decreasing dose, over time.

In certain embodiments, the methods of the present invention comprise administering to a subject in need thereof a therapeutically effective amount of a bispecific anti-CD20/anti-CD3 antibody, wherein administration of the bispecific anti-CD20/anti-CD3 antibody leads to increased inhibition of tumor cells. In certain embodiments, tumor cell population growth is inhibited by at least about 10%, about 20%, about 30%, about 40%, about 50%, about 60%, about 70% or about 80% as compared to an untreated subject. In certain embodiments, the administration of a bispecific anti-CD20/anti-CD3 antibody leads to increased tumor regression, tumor shrinkage and/or disappearance. In certain embodiments, the administration of a bispecific anti-CD20/anti-CD3 antibody leads to delay in tumor growth and development, e.g., tumor growth may be delayed by about 3 days, more than 3 days, about 7 days, more than 7 days, more than 15 days, more than 1 month, more than 3 months, more than 6 months, more than 1 year, more than 2 years, or more than 3 years as compared to an untreated subject. In certain embodiments, administration of a bispecific anti-CD20/anti-CD3 antibody prevents tumor recurrence and/or increases duration of survival of the subject, e.g., increases duration of survival by more than 15 days, more than 1 month, more than 3 months, more than 6 months, more than 12 months, more than 18 months, more than 24 months, more than 36 months, or more than 48 months than an untreated subject. In certain embodiments, administration of the bispecific anti-CD20/anti-CD3 antibody increases progression-free survival or overall survival. In certain embodiments, administration of a bispecific anti-CD20/anti-CD3 antibody increases response and duration of response in a subject, e.g., by more than 2%, more than 3%, more than 4%, more than 5%, more than 6%, more than 7%, more than 8%, more than 9%, more than 10%, more than 20%, more than 30%, more than 40% or more than 50% over an untreated subject. In certain embodiments, administration of a bispecific anti-CD20/anti-CD3 antibody to a subject with ALL leads to complete disappearance of all evidence of tumor cells (“complete response”). In certain embodiments, administration of a bispecific anti-CD20/anti-CD3 antibody to a subject with ALL leads to at least 30% or more decrease in tumor cells or tumor size (“partial response”). In certain embodiments, administration of a bispecific anti-CD20/anti-CD3 antibody to a subject with ALL leads to complete or partial disappearance of tumor cells/lesions including new measurable lesions. Tumor reduction can be measured by any of the methods known in the art, e.g., X-rays, positron emission tomography (PET), computed tomography (CT), magnetic resonance imaging (MRI), cytology, histology, or molecular genetic analyses.

### Antibodies and Antigen-Binding Fragments Thereof

According to certain exemplary embodiments of the present invention, the methods comprise administering a therapeutically effective amount of bispecific anti-CD20/anti-CD3 antibody or antigen-binding fragment thereof. The term “antibody,” as used herein, includes immunoglobulin molecules comprising four polypeptide chains, two heavy (H) chains and two light (L) chains inter-connected by disulfide bonds, as well as multimers thereof (e.g., IgM). In a typical antibody, each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (CO). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the invention, the FRs of the anti-IL-4R antibody (or antigen-binding portion thereof) may be identical to the human germline sequences, or may be naturally or artificially modified. An amino acid consensus sequence may be defined based on a side-by-side analysis of two or more CDRs.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAbfragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}- C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present invention may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

The term “antibody,” as used herein, also includes multispecific (e.g., bispecific) antibodies. A multispecific antibody or antigen-binding fragment of an antibody will typically comprise at least two different variable domains, wherein each variable domain is capable of specifically binding to a separate antigen or to a different epitope on the same antigen. Any multispecific antibody format may be adapted for use in the context of an antibody or antigen-binding fragment of an antibody of the present invention using routine techniques available in the art. Exemplary bispecific formats that can be used in the context of the present invention include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED) body, leucine zipper, Duobody, IgG1/IgG2, dual acting Fab (DAF)-IgG, and Mab² bispecific formats (see, e.g., Klein et al. 2012, mAbs 4:6, 1-11, and references cited therein, for a review of the foregoing formats). Bispecific antibodies can also be constructed using peptide/nucleic acid conjugation, e.g., wherein unnatural amino acids with orthogonal chemical reactivity are used to generate site-specific antibody-oligonucleotide conjugates which then self-assemble into multimeric complexes with defined composition, valency and geometry. (See, e.g., Kazane et al., J. Am. Chem. Soc. [Epub: Dec. 4, 2012]).

The antibodies used in the methods of the present invention may be human antibodies. The term “human antibody,” as used herein, is intended to include antibodies having variable and constant regions derived from human germline immunoglobulin sequences. The human antibodies of the invention may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

The antibodies used in the methods of the present invention may be recombinant human antibodies. The term “recombinant human antibody,” as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

According to certain embodiments, the antibodies used in the methods of the present invention specifically bind CD20 and CD3. The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” CD20 and CD3, as used in the context of the present invention, includes antibodies that bind CD20 and CD3 or portion thereof with a K_{D} of less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM or less than about 0.5 nM, as measured in a surface plasmon resonance assay. An isolated antibody that specifically binds human CD20 and CD3 may, however, have cross-reactivity to other antigens, such as CD20 and CD3 molecules from other (non-human) species.

According to certain exemplary embodiments, the methods of the present invention comprise the use of REGN1979, or a bioequivalent thereof. The term “bioequivalent”, as used herein, refers to bispecific anti-CD20/anti-CD3 antibodies or CD20 and/or CD3 binding proteins or fragments thereof that are pharmaceutical equivalents or pharmaceutical alternatives whose rate and/or extent of absorption do not show a significant difference with that of REGN1979 when administered at the same molar dose under similar experimental conditions, either single dose or multiple dose. In the context of the invention, the term refers to antigen-binding proteins that bind to CD20 and/or CD3 which do not have clinically meaningful differences with REGN1979 in their safety, purity and/or potency.

The anti-CD3/anti-CD20 bispecific antibody used in the context of the methods of the present invention may have pH-dependent binding characteristics. For example, an anti-CD3 antibody of the present invention may exhibit reduced binding to CD3 at acidic pH as compared to neutral pH. Alternatively, anti-CD3 antibodies of the invention may exhibit enhanced binding to CD3 at acidic pH as compared to neutral pH. For example, an anti-CD20 antibody of the present invention may exhibit reduced binding to CD3 at acidic pH as compared to neutral pH. Alternatively, anti-C20 antibodies of the invention may exhibit enhanced binding to CD3 at acidic pH as compared to neutral pH. The expression “acidic pH” includes pH values less than about 6.2, e.g., about 6.0, 5.95, 5.9, 5.85, 5.8, 5.75, 5.7, 5.65, 5.6, 5.55, 5.5, 5.45, 5.4, 5.35, 5.3, 5.25, 5.2, 5.15, 5.1, 5.05, 5.0, or less. As used herein, the expression “neutral pH” means a pH of about 7.0 to about 7.4. The expression “neutral pH” includes pH values of about 7.0, 7.05, 7.1, 7.15, 7.2, 7.25, 7.3, 7.35, and 7.4.

In certain instances, “reduced binding . . . at acidic pH as compared to neutral pH” is expressed in terms of a ratio of the K_{D} value of the antibody binding to its antigen at acidic pH to the K_{D} value of the antibody binding to its antigen at neutral pH (or vice versa). For example, an antibody or antigen-binding fragment thereof may be regarded as exhibiting “reduced binding to CD3 (or CD20) at acidic pH as compared to neutral pH” for purposes of the present invention if the antibody or antigen-binding fragment thereof exhibits an acidic/neutral K_{D} ratio of about 3.0 or greater. In certain exemplary embodiments, the acidic/neutral K_{D} ratio for an antibody or antigen-binding fragment of the present invention can be about 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 20.0, 25.0, 30.0, 40.0, 50.0, 60.0, 70.0, 100.0, or greater.

Antibodies with pH-dependent binding characteristics may be obtained, e.g., by screening a population of antibodies for reduced (or enhanced) binding to a particular antigen at acidic pH as compared to neutral pH. Additionally, modifications of the antigen-binding domain at the amino acid level may yield antibodies with pH-dependent characteristics. For example, by substituting one or more amino acids of an antigen-binding domain (e.g., within a CDR) with a histidine residue, an antibody with reduced antigen-binding at acidic pH relative to neutral pH may be obtained. As used herein, the expression “acidic pH” means a pH of 6.0 or less.

### Bispecific Anti-CD20/Anti-CD3 Antibodies

According to certain exemplary embodiments of the present invention, the methods comprise administering a therapeutically effective amount of a bispecific antibody that specifically binds CD3 and CD20. Such antibodies may be referred to herein as, e.g., “anti-CD20/anti-CD3,” or “anti-CD20×CD3” or “CD20×CD3” bispecific antibodies, or other similar terminology.

As used herein, the expression “bispecific antibody” refers to an immunoglobulin protein comprising at least a first antigen-binding domain and a second antigen-binding domain. In the context of the present invention, the first antigen-binding domain specifically binds a first antigen (e.g., CD20), and the second antigen-binding domain specifically binds a second, distinct antigen (e.g., CD3). Each antigen-binding domain of a bispecific antibody comprises a heavy chain variable domain (HCVR) and a light chain variable domain (LCVR), each comprising three CDRs. In the context of a bispecific antibody, the CDRs of the first antigen-binding domain may be designated with the prefix “A” and the CDRs of the second antigen-binding domain may be designated with the prefix “B”. Thus, the CDRs of the first antigen-binding domain may be referred to herein as A-HCDR1, A-HCDR2, and A-HCDR3; and the CDRs of the second antigen-binding domain may be referred to herein as B-HCDR1, B-HCDR2, and B-HCDR3.

The first antigen-binding domain and the second antigen-binding domain are each connected to a separate multimerizing domain. As used herein, a “multimerizing domain” is any macromolecule, protein, polypeptide, peptide, or amino acid that has the ability to associate with a second multimerizing domain of the same or similar structure or constitution. In the context of the present invention, the multimerizing component is an Fc portion of an immunoglobulin (comprising a C_{H}2-C_{H}3 domain), e.g., an Fc domain of an IgG selected from the isotypes IgG1, IgG2, IgG3, and IgG4, as well as any allotype within each isotype group.

Bispecific antibodies of the present invention typically comprise two multimerizing domains, e.g., two Fc domains that are each individually part of a separate antibody heavy chain. The first and second multimerizing domains may be of the same IgG isotype such as, e.g., IgG1/IgG1, IgG2/IgG2, IgG4/IgG4. Alternatively, the first and second multimerizing domains may be of different IgG isotypes such as, e.g., IgG1/IgG2, IgG1/IgG4, IgG2/IgG4, etc.

Any bispecific antibody format or technology may be used to make the bispecific antigen-binding molecules of the present invention. For example, an antibody or fragment thereof having a first antigen binding specificity can be functionally linked (e.g., by chemical coupling, genetic fusion, noncovalent association or otherwise) to one or more other molecular entities, such as another antibody or antibody fragment having a second antigen-binding specificity to produce a bispecific antigen-binding molecule. Specific exemplary bispecific formats that can be used in the context of the present invention include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED)body, leucine zipper, Duobody, IgG1/IgG2, dual acting Fab (DAF)-IgG, and Mab² bispecific formats (see, e.g., Klein et al. 2012, mAbs 4:6, 1-11, and references cited therein, for a review of the foregoing formats).

In the context of bispecific antibodies of the present invention, Fc domains may comprise one or more amino acid changes (e.g., insertions, deletions or substitutions) as compared to the wild-type, naturally occurring version of the Fc domain. For example, the invention includes bispecific antigen-binding molecules comprising one or more modifications in the Fc domain that results in a modified Fc domain having a modified binding interaction (e.g., enhanced or diminished) between Fc and FcRn. In one embodiment, the bispecific antigen-binding molecule comprises a modification in a C_{H}2 or a C_{H}3 region, wherein the modification increases the affinity of the Fc domain to FcRn in an acidic environment (e.g., in an endosome where pH ranges from about 5.5 to about 6.0). Non-limiting examples of such Fc modifications are disclosed in US Patent Publication No. 20150266966, incorporated herein in its entirety.

The present invention also includes bispecific antibodies comprising a first C_{H}3 domain and a second Ig C_{H}3 domain, wherein the first and second Ig C_{H}3 domains differ from one another by at least one amino acid, and wherein at least one amino acid difference reduces binding of the bispecific antibody to Protein A as compared to a bi-specific antibody lacking the amino acid difference. In one embodiment, the first Ig C_{H}3 domain binds Protein A and the second Ig C_{H}3 domain contains a mutation that reduces or abolishes Protein A binding such as an H95R modification (by IMGT exon numbering; H435R by EU numbering). The second C_{H}3 may further comprise a Y96F modification (by IMGT; Y436F by EU). Further modifications that may be found within the second C_{H}3 include: D16E, L18M, N44S, K52N, V57M, and V821 (by IMGT; D356E, L358M, N384S, K392N, V397M, and V4221 by EU) in the case of IgG1 antibodies; N44S, K52N, and V821 (IMGT; N384S, K392N, and V4221 by EU) in the case of IgG2 antibodies; and Q15R, N44S, K52N, V57M, R69K, E79Q, and V821 (by IMGT; Q355R, N384S, K392N, V397M, R409K, E419Q, and V4221 by EU) in the case of IgG4 antibodies.

In certain embodiments, the Fc domain may be chimeric, combining Fc sequences derived from more than one immunoglobulin isotype. For example, a chimeric Fc domain can comprise part or all of a C_{H}2 sequence derived from a human IgG1, human IgG2 or human IgG4 C_{H}2 region, and part or all of a C_{H}3 sequence derived from a human IgG1, human IgG2 or human IgG4. A chimeric Fc domain can also contain a chimeric hinge region. For example, a chimeric hinge may comprise an “upper hinge” sequence, derived from a human IgG1, a human IgG2 or a human IgG4 hinge region, combined with a “lower hinge” sequence, derived from a human IgG1, a human IgG2 or a human IgG4 hinge region. A particular example of a chimeric Fc domain that can be included in any of the antigen-binding molecules set forth herein comprises, from N- to C-terminus: [IgG4 C_{H}1]-[IgG4 upper hinge]-[IgG2 lower hinge]-[IgG4 C_{H}2]-[IgG4 C_{H}3]. Another example of a chimeric Fc domain that can be included in any of the antigen-binding molecules set forth herein comprises, from N- to C-terminus: [IgG1 C_{H}1]-[IgG1 upper hinge]-[IgG2 lower hinge]-[IgG4 C_{H}2] [IgG1 C_{H}3]. These and other examples of chimeric Fc domains that can be included in any of the antigen-binding molecules of the present invention are described in US Patent Publication No. 20140243504, which is herein incorporated in its entirety. Chimeric Fc domains having these general structural arrangements, and variants thereof, can have altered Fc receptor binding, which in turn affects Fc effector function. In particular embodiments, the Fc domain can comprise the amino acid sequence of SEQ ID NO:13 or SEQ ID NO:14.

According to certain exemplary embodiments of the present invention, the bispecific anti-CD20/anti-CD3 antibody, or antigen-binding fragment thereof comprises heavy chain variable regions (A-HCVR and B-HCVR), light chain variable region (LCVR), and/or complementarity determining regions (CDRs) comprising any of the amino acid sequences of the bispecific anti-CD20/anti-CD3 antibodies as set forth in US Patent Publication No. 20150266966. In certain exemplary embodiments, the bispecific anti-CD20/anti-CD3 antibody or antigen-binding fragment thereof that can be used in the context of the methods of the present invention comprises: (a) a first antigen-binding arm comprising the heavy chain complementarity determining regions (A-HCDR1, A-HCDR2 and A-HCDR3) of a heavy chain variable region (A-HCVR) comprising the amino acid sequence of SEQ ID NO: 1 and the light chain complementarity determining regions (LCDRs) of a light chain variable region (LCVR) comprising the amino acid sequence of SEQ ID NO: 2; and (b) a second antigen-binding arm comprising the heavy chain CDRs (B-HCDR1, B-HCDR2 and B-HCDR3) of a HCVR (B-HCVR) comprising the amino acid sequence of SEQ ID NO: 3 and the light chain CDRs of a LCVR comprising the amino acid sequence of SEQ ID NO: 2. According to certain embodiments, the A-HCDR1 comprises the amino acid sequence of SEQ ID NO: 4; the A-HCDR2 comprises the amino acid sequence of SEQ ID NO: 5; the A-HCDR3 comprises the amino acid sequence of SEQ ID NO: 6; the LCDR1 comprises the amino acid sequence of SEQ ID NO:7; the LCDR2 comprises the amino acid sequence of SEQ ID NO: 8; the LCDR3 comprises the amino acid sequence of SEQ ID NO: 9; the B-HCDR1 comprises the amino acid sequence of SEQ ID NO: 10; the B-HCDR2 comprises the amino acid sequence of SEQ ID NO: 11; and the B-HCDR3 comprises the amino acid sequence of SEQ ID NO: 12. In yet other embodiments, the bispecific anti-CD20/anti-CD3 antibody or antigen-binding fragment thereof comprises: (a) a first antigen-binding arm comprising a HCVR (A-HCVR) comprising SEQ ID NO: 1 and a LCVR comprising SEQ ID NO: 2; and (b) a second antigen-binding arm comprising a HCVR (B-HCVR) comprising SEQ ID NO: 3 and a LCVR comprising SEQ ID NO: 2.

Other bispecific anti-CD20/anti-CD3 antibodies that can be used in the context of the methods of the present invention include, e.g., any of the antibodies as set forth in US Patent Publication Nos. 20140088295 and 20150166661. An exemplary bispecific anti-CD20/anti-CD3 antibody that can be used in the context of the methods of the present invention is the bispecific anti-CD20/anti-CD3 antibody known as REGN1979 or bsAB1.

### Combination Therapies

In certain embodiments, the methods of the present invention comprise administration of a second therapeutic agent wherein the second therapeutic agent is an anti-cancer drug. As used herein, “anti-cancer drug” means any agent useful to treat cancer including, but not limited to, cytotoxins and agents such as antimetabolites, alkylating agents, anthracyclines, antibiotics, antimitotic agents, procarbazine, hydroxyurea, asparaginase, corticosteroids, mytotane (O,P′-(DDD)), biologics (e.g., antibodies and interferons) and radioactive agents. As used herein, “a cytotoxin or cytotoxic agent”, also refers to a chemotherapeutic agent and means any agent that is detrimental to cells. Examples include Taxol® (paclitaxel), temozolamide, cytochalasin B, gramicidin D, ethidium bromide, emetine, cisplatin, mitomycin, etoposide, tenoposide, vincristine, vinbiastine, coichicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof.

In certain embodiments, the methods of the present invention comprise administration of a second therapeutic agent selected from the group consisting of radiation, surgery, a cancer vaccine, a PD-1 inhibitor (e.g., an anti-PD-1 antibody), a PD-L1 inhibitor (e.g., an anti-PD-L1 antibody), a LAG-3 inhibitor, a CTLA-4 inhibitor (e.g., ipilimumab), a TIM3 inhibitor, a BTLA inhibitor, a TIGIT inhibitor, a CD47 inhibitor, an antagonist of another T-cell co-inhibitor or ligand (e.g., an antibody to CD-28, 2B4, LY108, LAIR1, ICOS, CD160 or VISTA), an indoleamine-2,3-dioxygenase (IDO) inhibitor, a vascular endothelial growth factor (VEGF) antagonist [e.g., a “VEGF-Trap” such as aflibercept or other VEGF-inhibiting fusion protein as set forth in U.S. Pat. No. 7,087,411, or an anti-VEGF antibody or antigen binding fragment thereof (e.g., bevacizumab, or ranibizumab) or a small molecule kinase inhibitor of VEGF receptor (e.g., sunitinib, sorafenib, or pazopanib)], an Ang2 inhibitor (e.g., nesvacumab), a transforming growth factor beta (TGFβ) inhibitor, an epidermal growth factor receptor (EGFR) inhibitor (e.g., erlotinib, cetuximab), an agonist to a co-stimulatory receptor (e.g., an agonist to glucocorticoid-induced TNFR-related protein), an antibody to a tumor-specific antigen (e.g., CA9, CA125, melanoma-associated antigen 3 (MAGES), carcinoembryonic antigen (CEA), vimentin, tumor-M2-PK, prostate-specific antigen (PSA), mucin-1, MART-1, and CA19-9), a vaccine (e.g., Bacillus Calmette-Guerin, a cancer vaccine), an adjuvant to increase antigen presentation (e.g., granulocyte-macrophage colony-stimulating factor), a cytotoxin, a chemotherapeutic agent (e.g., dacarbazine, temozolomide, cyclophosphamide, docetaxel, doxorubicin, daunorubicin, cisplatin, carboplatin, gemcitabine, methotrexate, mitoxantrone, oxaliplatin, paclitaxel, and vincristine), radiotherapy, an IL-6R inhibitor (e.g., sarilumab), an IL-4R inhibitor (e.g., dupilumab), an IL-10 inhibitor, a cytokine such as IL-2, IL-7, IL-21, and IL-15, an antibody-drug conjugate (ADC) (e.g., anti-CD19-DM4 ADC, and anti-DS6-DM4 ADC), chimeric antigen receptor T cells (e.g., CD19-targeted T cells), an anti-inflammatory drug (e.g., corticosteroids, and non-steroidal anti-inflammatory drugs), and a dietary supplement such as anti-oxidants.

In certain embodiments, the methods of the invention comprise administering an anti-CD20/anti-CD3 bispecific antibody in combination with radiation therapy to generate long-term durable anti-tumor responses and/or enhance survival of patients with cancer (e.g., ALL).

In some embodiments, the methods of the invention comprise administering radiation therapy prior to, concomitantly or after administering a bispecific anti-CD20/anti-CD3 antibody to a cancer patient. For example, radiation therapy may be administered in one or more doses to tumor lesions after administration of one or more doses of the antibody. In some embodiments, radiation therapy may be administered locally to a tumor lesion to enhance the local immunogenicity of a patient's tumor (adjuvinating radiation) and/or to kill tumor cells (ablative radiation) after systemic administration of a bispecific anti-CD20/anti-CD3 antibody. In certain embodiments, the antibodies may be administered in combination with radiation therapy and a chemotherapeutic agent (e.g., temozolomide or cyclophosphamide) or a VEGF antagonist (e.g., aflibercept).

### Pharmaceutical Compositions and Administration

The pharmaceutical compositions of the invention may be formulated with suitable carriers, excipients, and other agents that provide suitable transfer, delivery, tolerance, and the like. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

Various delivery systems are known and can be used to administer the pharmaceutical composition of the invention, e.g., encapsulation in liposomes, microparticles, microcapsules, recombinant cells capable of expressing the mutant viruses, receptor mediated endocytosis (see, e.g., Wu et al., 1987, J. Biol. Chem. 262: 4429-4432). Methods of administration include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents.

A pharmaceutical composition of the present invention can be delivered subcutaneously or intravenously with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

In certain situations, the pharmaceutical composition can be delivered in a controlled release system. In one embodiment, a pump may be used. In another embodiment, polymeric materials can be used; see, Medical Applications of Controlled Release, Langer and Wise (eds.), 1974, CRC Pres., Boca Raton, Fla. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by known methods. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc.

### Administration Regimens

The present invention includes methods comprising administering to a subject a bispecific anti-CD20/anti-CD3 antibody at a dosing frequency of about four times a week, twice a week, once a week, once every two weeks, once every three weeks, once every four weeks, once every five weeks, once every six weeks, once every eight weeks, once every twelve weeks, or less frequently so long as a therapeutic response is achieved.

According to certain embodiments of the present invention, multiple doses of a bispecific anti-CD20/anti-CD3 antibody may be administered to a subject over a defined time course. The methods according to this aspect of the invention comprise sequentially administering to a subject one or more doses of a bispecific anti-CD20/anti-CD3 antibody. As used herein, “sequentially administering” means that each dose of the antibody is administered to the subject at a different point in time, e.g., on different days separated by a predetermined interval (e.g., hours, days, weeks or months). The present invention includes methods which comprise sequentially administering to the patient a single initial dose of a bispecific anti-CD20/anti-CD3 antibody, followed by one or more secondary doses of the bispecific antibody, and optionally followed by one or more tertiary doses of the bispecific antibody.

The terms “initial dose,” “secondary doses,” and “tertiary doses,” refer to the temporal sequence of administration. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of the bispecific anti-CD20/anti-CD3 antibody. In certain embodiments, however, the amount contained in the initial, secondary and/or tertiary doses varies from one another (e.g., adjusted up or down as appropriate) during the course of treatment. In certain embodiments, one or more (e.g., 1, 2, 3, 4, or 5) doses are administered at the beginning of the treatment regimen as “loading doses” followed by subsequent doses that are administered on a less frequent basis (e.g., “maintenance doses”). For example, bispecific anti-CD20/anti-CD3 antibody may be administered to a patient with ALL at a loading dose of, e.g., about 0.1 to 10 mg/kg followed by one or more maintenance doses of, e.g., about 0.1 to 10 mg/kg of the patient's body weight.

In one exemplary embodiment of the present invention, each secondary and/or tertiary dose is administered ½ to 14 (e.g., ½, 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, or more) weeks after the immediately preceding dose. The phrase “the immediately preceding dose,” as used herein, means, in a sequence of multiple administrations, the dose of bispecific anti-CD20/anti-CD3 antibody which is administered to a patient prior to the administration of the very next dose in the sequence with no intervening doses.

The methods according to this aspect of the invention may comprise administering to a patient any number of secondary and/or tertiary doses of bispecific anti-CD20/anti-CD3 antibody. For example, in certain embodiments, only a single secondary dose is administered to the patient. In other embodiments, two or more (e.g., 2, 3, 4, 5, 6, 7, 8, or more secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 2 weeks after the immediately preceding dose. Similarly, in embodiments involving multiple tertiary doses, each tertiary dose may be administered at the same frequency as the other tertiary doses. For example, each tertiary dose may be administered to the patient 2 to 4 weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

In certain embodiments, one or more doses of bispecific anti-CD20/anti-CD3 antibody are administered at the beginning of a treatment regimen as “induction doses” on a more frequent basis (twice a week, once a week or once in 2 weeks) followed by subsequent doses (“consolidation doses” or “maintenance doses”) that are administered on a less frequent basis (e.g., once in 4-12 weeks).

The present invention includes methods comprising administration of a bispecific anti-CD20/anti-CD3 antibody, to a patient to treat ALL. In some embodiments, the present methods comprise administering one or more doses of a bispecific anti-CD20/anti-CD3 antibody. In some embodiments, one or more doses of about 0.1 mg/kg to about 10 mg/kg of the bispecific antibody to inhibit tumor growth and/or to prevent tumor recurrence in a subject with ALL. In some embodiments, the bispecific anti-CD20/anti-CD3 antibody is administered at one or more doses resulting in increased anti-tumor efficacy (e.g., greater inhibition of tumor growth, increased prevention of tumor recurrence as compared to an untreated subject or a subject administered with either antibody as monotherapy).

### Dosage

The amount of bispecific anti-CD20/anti-CD3 antibody administered to a subject according to the methods of the present invention is, generally, a therapeutically effective amount. As used herein, the phrase “therapeutically effective amount” means an amount of bispecific anti-CD20/anti-CD3 antibody that results in one or more of: (a) a reduction in the severity or duration of a symptom of ALL; (b) inhibition of tumor growth, or an increase in tumor necrosis, tumor shrinkage and/or tumor disappearance; (c) delay in tumor growth and development; (d) inhibit or retard or stop tumor metastasis; (e) prevention of recurrence of tumor growth; (f) increase in survival of a subject with ALL; and/or (g) a reduction in the use or need for conventional anti-cancer therapy (e.g., reduced or eliminated use of chemotherapeutic or cytotoxic agents) as compared to an untreated subject or a subject administered with either antibody as monotherapy.

Alternatively, a single, first dose is administered at a low dose, such as 10 μg, or 30 μg, or 100 μg, and then after a period of time, a second dose is administered at a higher dose, such as two or three times higher than the first dose, in order to prevent, reduce or ameliorate cytokine storm in a patient. By reducing “cytokine storm” in a patient refers to reducing the effect of a cytokine cascade or hypercytokinemia, wherein such negative immune reaction may be caused by, but is not limited to a positive feedback loop between cytokines and white blood cells, and/or highly elevated levels of various cytokines.

According to Example 2, herein, a first (initial) dose of the bispecific antigen-binding molecule of the invention (e.g. Ab 1, also known as bsAB1 or REGN1979) is administered, followed by a subsequent second dose after a period of time, wherein the second dose exceeds (is greater than) the first dose. In some embodiments, the second dose is about 2 times greater than, or about 3 times greater than the first dose. In another embodiment, the bispecific antigen-binding molecule is administered at the first dose weekly for consecutive weeks, such as four (4) consecutive weeks. In another embodiment, the first dose is administered weekly followed by monthly doses for an additional period of time (or a designated number of monthly doses). In some embodiments, following the designated dosing regime for the first dose, the second dose is administered weekly followed by monthly doses for an additional period of time. In some embodiments, the first (initial) dose is 10 μg, and the second dose is 30 μg. In some embodiments, the first (initial) dose is 30 μg, and the second dose is 100 μg. In other embodiments, the first (initial) dose is 100 μg, and the second dose is 300 μg. In other embodiments, the first (initial) dose is 300 μg, and the second dose is 1000 μg. In other embodiments, the first (initial) dose is 1000 μg, and the second dose is 2000 μg. In other embodiments, the first (initial) dose is 1000 μg, and the second dose is 3000 μg. In other embodiments, the first (initial) dose is 1000 μg, and the second dose is 4000 μg. In other embodiments, the first (initial) dose is 1000 μg, and the second dose is 5000 μg. In other embodiments, the first (initial) dose is 2000 μg, and the second dose is 3000 μg. In other embodiments, the first (initial) dose is 3000 μg, and the second dose is 4000 μg. In other embodiments, the first (initial) dose is 4000 μg, and the second dose is 5000 μg. In other embodiments, the first (initial) dose is 5000 μg, and the second dose is 6000 μg. In other embodiments, the first (initial) dose is 6000 μg, and the second dose is 7000 μg. In other embodiments, the first (initial) dose is 7000 μg, and the second dose is 8000 μg.

In the case of a bispecific anti-CD20/anti-CD3 antibody, a therapeutically effective amount can be from about 10 micrograms (mcg) to about 8000 mcg, e.g., about 10 mcg, about 20 mcg, about 30 mcg, about 50 mcg, about 70 mcg, about 100 mcg, about 120 mcg, about 150 mcg, about 200 mcg, about 250 mcg, about 300 mcg, about 350 mcg, about 400 mcg, about 450 mcg, about 500 mcg, about 550 mcg, about 600 mcg, about 700 mcg, about 800 mcg, about 900 mcg, about 1000 mcg, about 1050 mcg, about 1100 mcg, about 1500 mcg, about 1700 mcg, about 2000 mcg, about 2050 mcg, about 2100 mcg, about 2200 mcg, about 2500 mcg, about 2700 mcg, about 2800 mcg, about 2900 mcg, about 3000 mcg, about 4000 mcg, about 5000 mcg, about 6000 mcg, about 7000 mcg, or about 8000 mcg of the bispecific anti-CD20/anti-CD3 antibody.

The amount of bispecific anti-CD20/anti-CD3 antibody contained within the individual doses may be expressed in terms of milligrams of antibody per kilogram of subject body weight (i.e., mg/kg). In certain embodiments, a bispecific anti-CD20/anti-CD3 antibody used in the methods of the present invention may be administered to a subject at a dose of about 0.0001 to about 100 mg/kg of subject body weight. In certain embodiments, bispecific anti-CD20/anti-CD3 antibody used in the methods of the present invention may be administered to a subject at a dose of about 100 mg/kg, of about 90 mg/kg, of about 80 mg/kg, about 70 mg/kg, of about 60 mg/kg, of about 50 mg/kg, of about 40 mg/kg, of about 30 mg/kg, of about 20 mg/kg, of about 10 mg/kg, of about 9 mg/kg, of about 8 mg/kg, of about 7 mg/kg, of about 6 mg/kg, of about 5 mg/kg, of about 4 mg/kg, of about 3 mg/kg, of about 2 mg/kg, of about 1 mg/kg, of about 0.9 mg/kg, of about 0.8 mg/kg, of about 0.7 mg/kg, of about 0.6 mg/kg, of about 0.5 mg/kg, of about 0.4 mg/kg, of about 0.3 mg/kg, of about 0.2 mg/kg, of about 0.1 mg/kg, of about 0.08 mg/kg, of about 0.06 mg/kg, of about 0.04 mg/kg, of about 0.03 mg/kg, of about 0.02 mg/kg, of about 0.01 mg/kg, of about 0.001 mg/kg, of about 0.0001 mg/kg or less. For example, the bispecific anti-CD20/anti-CD3 antibody may be administered at a dose of about 0.1 mg/kg to about 10 mg/kg of a patient's body weight.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1. Treatment with CD3×CD20 Bispecific Antibody is More Effective than Anti-CD20+ Antibody in NSG Mice with Established Raji Tumors

The efficacy of selected anti-CD3×CD20 bispecific antibodies in reducing established tumors in NSG mice was assessed. NSG mice (NOD/LtSz-scid/IL2Rγnull mice; Jackson Laboratories) were subcutaneously co-implanted with Raji tumor cells (2×10⁶) and human PBMCs (5×10⁶)(at Day −14). Tumors were allowed to establish in the host for 14 days prior to treatment.

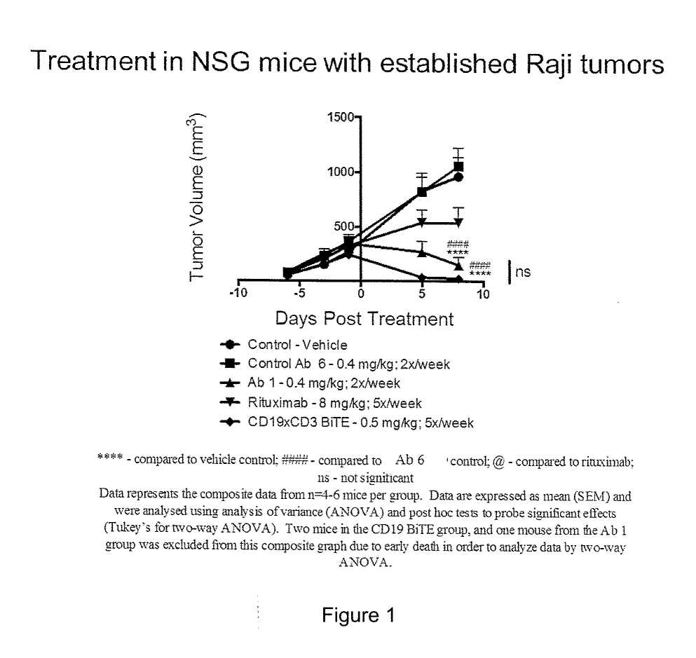

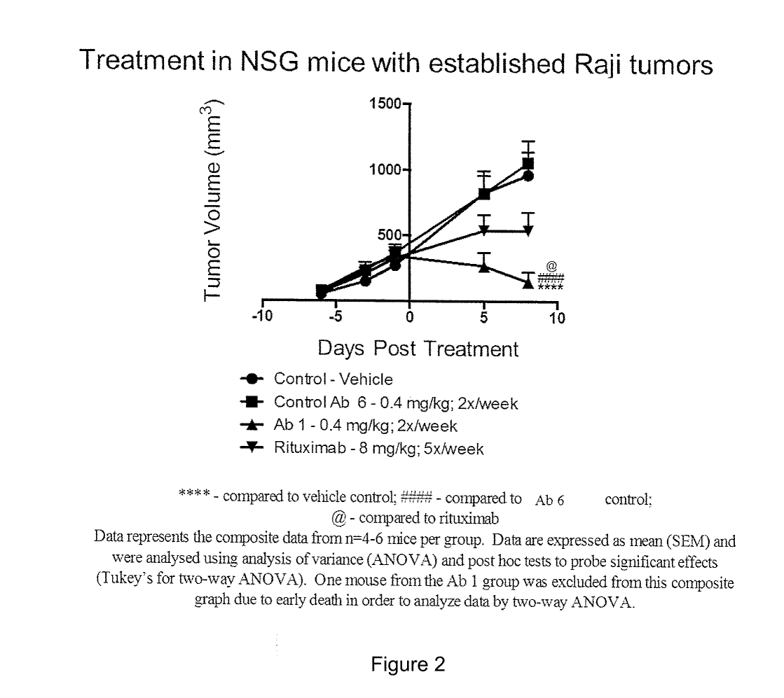

The CD20×CD3 bispecific Ab1 (also known as bsAB1 and REGN1979) (dosed at 0.4 mg/kg; 2×/week i.p.) was comparable to the CD19×CD3 BiTE (dosed at 0.5 mg/kg; 5×/week i.v.) (FIG. 1) and superior to rituximab therapy (dosed at 8 mg/kg; 5×/week i.p.) (FIG. 2) in suppressing established Raji tumors, thereby demonstrating that Ab1 (also known as bsAB1 and REGN1979) was effective at treating mammals with large lymphoma masses greater than 0.5 cm in volume.

### Example 2: Clinical Trial of Anti-CD20×CD3 Antibody in Patients with Acute Lymphoblastic Leukemia

This study is an open-label, multicenter, dose escalation study with multiple dose escalation and expansion arms to investigate the efficacy, safety, and tolerability of anti-CD20/anti-CD3 bispecific antibody in adult patients with acute lymphoblastic leukemia.

The exemplary bispecific anti-CD20/anti-CD3 antibody used in this Example is REGN1979 (described in Example 1 herein).

The primary objective of the study is to assess safety, tolerability and dose-limiting toxicity (DLT) of REGN1979 in patients with Acute Lymphoblastic Leukemia (ALL).

The secondary objectives of the study are: (i) to determine a recommended dose for: REGN1979 in patients with ALL; (ii) to characterize the pharmacokinetic (PK) profile of REGN1979; (iii) to assess the immunogenicity of REGN1979; and (iv) to study the preliminary antitumor activity of REGN1979 in ALL, as measured by overall response rate, minimal residual disease (MRD) in patients with bone marrow disease at baseline, duration of response, progression-free survival, median, and rate at 6 and 12 months.

Additional objectives are to evaluate biomarkers that may correlate with mechanism of action, observed toxicity, and potential antitumor activity including, but not limited to: cytokine profiling; peripheral blood B- and T-cell subsets and immune phenotyping; changes in gene expression in peripheral blood; and serum immunoglobulin.

### Study Population

The target population includes patients with ALL for whom no standard of care options exist.

### Inclusion Criteria for Acute Lymphoblastic Leukemia Study Arms

A patient must meet the following criteria to be eligible for inclusion in the study: (1) Documented relapsed or refractory CD20+(defined as CD20 expression by flow cytometry on ≧20% of leukemic lymphoblasts) B-lineage ALL after at least induction and 1 cycle of consolidation chemotherapy a. Patients with Philadelphia chromosome positive ALL are required to have failed or be intolerant to at least 1 tyrosine-kinase inhibitor NOTE: Patients with chronic myeloid leukemia (CML) blast crisis with lymphoid phenotype are allowed, provided they meet inclusion criterion #1; (2) Age 8 years; (3) ECOG performance status 2; (4) CNS negative disease, confirmed by lumbar puncture, within 28 days of starting study drug; (5) Adequate bone marrow function documented by: a. Platelet counts ≧10×10⁹/Lb. Hb level ≧7 g/dL c. Absolute phagocyte count ≧0.5×10⁹/L (phagocytes: neutrophils, bands and monocytes); (6) Adequate hepatic function:

Total bilirubin×ULN (3×ULN if liver involvement) b. Transaminases×ULN (5×ULN if liver involvement) c. Alkaline phosphatase×ULN (5×ULN if liver involvement) (NOTE: Patients with Gilbert's syndrome do not need to meet this requirement provided their total bilirubin is unchanged from their baseline. NOTE: Patients may be considered for enrollment if, in the opinion of the investigator, the abnormal laboratory results are due to current underlying malignancy. In such cases, the investigator must discuss the eligibility with the sponsor and receive approval for enrollment in writing); (7) Serum creatinine×ULN or calculated creatinine clearance by Cockcroft-Gault ≧50 mL/min (NOTE: Patients with creatinine clearance by Cockcroft-Gault that does not meet criteria may be considered for enrollment if a measured creatinine clearance (based on 24-hour urine or other reliable method) is 50 mL/min. NOTE: Patients may be considered for enrollment if, in the opinion of the investigator, the abnormal laboratory results are due to current underlying malignancy. In such cases, the investigator must discuss the eligibility with the sponsor and receive approval for enrollment in writing); (8) No sign of acute or chronic graft versus host disease (GvHD) and no anti-GvHD medication within 14 days prior to initiation of study drug(s); (9) Normal cardiac ejection fraction by pretreatment MUGA or echocardiogram within 4 weeks prior to enrollment within the normal range of values for the institution; (10) Willing and able to comply with clinic visits and study-related procedures; and (11) Provide signed informed consent.

### Exclusion Criteria for Acute Lymphoblastic Leukemia Treatment Arms

A patient who meets any of the following criteria will be excluded from the study: (1) History of or current relevant CNS pathology such as a. Epilepsy, seizure, paresis, aphasia, apoplexia, severe brain injuries, cerebellar disease, organic brain syndrome, psychosis, or b. Evidence for presence of inflammatory lesions and/or vasculitis on cerebral MRI during screening; (2) Burkitt's leukemia; (3) Current testicular involvement of leukemia; (4) Ongoing or recent (within 2 years) evidence of significant autoimmune disease (with the exception of GvHD) that required treatment with systemic immunosuppressive treatments, which may suggest risk for iAEs; (5) Standard anti-leukemia chemotherapy (nonbiologic) or radiotherapy less than 14 days prior to first administration of study drug(s); (6) Treatment with an investigational nonbiologic agent less than 14 days prior to first administration of study drug(s); (7) Treatment with rituximab, immune modulating agents or other investigational or commercial biologic agent less than 14 days prior to first administration of study drug. (Examples of immune modulating agents include blockers of CTLA-4, 4-1 BB (CD137), LAGS, OX-40, therapeutic vaccines, or cytokine treatments); (8) Treatment with alemtuzumab, less than 12 weeks prior to first administration of study drug(s); (9) Prior allogeneic stem cell transplantation within 3 months of treatment; (10) Concurrent active malignancy for which the patient is receiving treatment; (11) Evidence of significant concurrent disease or medical condition that could interfere with the conduct of the study, or put the patient at significant risk including, but not limited to, significant cardiovascular disease (eg, New York Heart Association Class III or IV cardiac disease, myocardial infarction within 6 months prior to screening, unstable arrhythmias or unstable angina) and/or significant pulmonary disease (eg, obstructive pulmonary disease and history of symptomatic bronchospasm); (12) Known active bacterial, viral, fungal, mycobacterial or other infection or any major episode of infection requiring hospitalization or treatment with IV anti-infectives within 14 days prior to first administration of study drug(s); (13) Infection with HIV or active infection with HBV or HCV; (14) History of pneumonitis within the last 5 years; (15) History of allergic reactions attributed to compounds of similar chemical or biologic composition of study drug(s); (16) History of hypersensitivity to any compound in the tetracycline antibiotics group (precaution due to potential presence of trace components in study drug material); (17) Known hypersensitivity to both allopurinol and rasburicase; (18) Pregnant or breastfeeding women; and (19) Sexually active men or women of childbearing potential who are unwilling to practice adequate contraception during the study and up to 6 months after discontinuation of study medication.

### Study Design

This is an open-label, multicenter, dose escalation study with multiple dose escalation and expansion arms.

Patients are assigned to one of the following cohorts:
Multiple dose escalation cohorts
2 expansion cohorts: a) relapsed/refractory ALL, and b) minimal residual disease-positive (MRD+) ALL
   REGN1979 in Patients with ALL

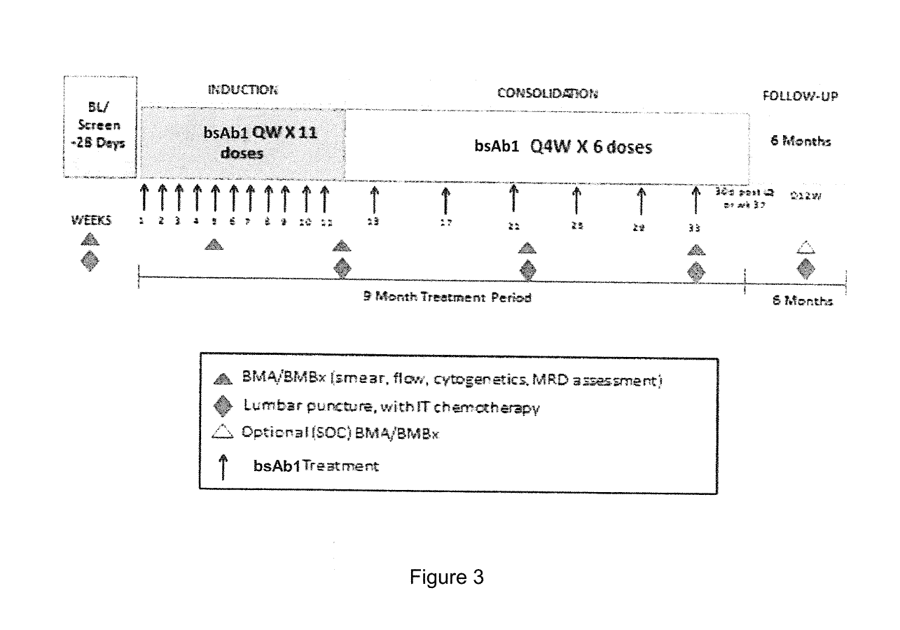

FIG. 3 shows REGN1979 treatment schedule for patients with ALL. Patients are assigned a DL that will consist of an initial starting dose followed by a subsequent higher dose, provided the initial starting dose was tolerated (Table 1).

TABLE 1
Dose levels for REGN1979

| | Initial dose | Subsequent dose |
|---|---|---|
| Dose Level | (flat mcg) | (flat mcg) |
| DL-1 | 10 | 30 |
| DL1 | 30 | 100 |
| DL2 | 100 | 300 |
| DL3 | 300 | 1000 |
| DL4 | 1000 | 2000 |
| DL5 | 1000 | 3000 |
| DL6 | 1000 | 4000 |
| DL7 | 1000 | 5000 |

Intravenous REGN1979 is administered weekly for 11 doses, followed by treatment every 4 weeks (04W) starting at week 13, for 6 additional doses. Patients are followed for an additional 6 months after completion of REGN1979 treatment.

### Starting Doses

REGN1979: The starting DL of REGN1979 in patients with ALL is based on the safety observed. The starting dose of the initial DL in patients with ALL will be at least 10 times lower than the initial dose of the DL that has cleared safety; however, the starting DL will not be lower than DL1. REGN1979 expansions in relapsed/refractory ALL and MRD-positive ALL is determined in the ALL dose escalation arm.

### Dose Escalation

In all arms and all cohorts, dose escalation rules will follow a traditional 3+3 dose escalation design, enrolling between 3 and 6 patients per cohort.

The dose limiting toxicity (DLT) observation period is defined as the first 28 days of treatment for all cohorts in all arms. Any of the following events occurring in the first 28 days of treatment (and considered to be related to study treatment by the investigator) is considered a DLT: grade uveitis, grade 4 neutropenia, grade 4 thrombocytopenia, and grade febrile neutropenia.

The maximum tolerated dose (MTD) is determined based on observed toxicity during the DLT observation period, and is defined as the dose level (DL) immediately below the level at which dosing is stopped due to the occurrence of DLTs in 2 or more patients. If the dose escalation portion of an arm is not stopped due to the occurrence of a DLT, it will be considered that the MTD has not been determined.

An optimal biological dose is also determined based on observed safety and tolerability, PK, PD, and preliminary antitumor activity.

The recommended dose for the expansion arms is determined based on review of the data used to determine the MTD and/or optimal biological dose.

### Study Duration

The study treatment period is from 6 to 12 months, depending on how an individual patient responds to treatment. The follow-up period is 6 months for all patients.

Sample size: Exact number of patients enrolled depend on the occurrence of protocol-defined DLTs and the number of DLs that will open. Sample size for REGN1979 in patients with ALL is up to 42 patients (depending on which DL this arm opens at). Each expansion cohort will enroll 20 patients, for a total of 180 patients.

### Study Treatments and Administration

REGN1979 is supplied as a liquid in sterile, single-use vials. Each vial contains a withdrawable volume of 1 mL of REGN1979 at a concentration of 2 mg/mL. A pharmacist or other qualified individual is identified at each site to prepare REGN1979 for administration. The dose(s) received are according to dose level cohort assignment. The dose administered at each dose level is a flat dose and not dependent on patient weight or body surface area. Each dose of REGN1979 is administered by intravenous (IV) infusion over at least 60 minutes. The infusion time may be extended to up to 4 hours, per the physician's clinical judgment. Additionally, the investigator may choose to split the dose into 2 separate infusions over 2 (preferably consecutive) days.

Premedication with dexamethasone at least 1 hour prior to infusion is required prior to administration of REGN1979 at doses of 300 mcg or higher. At least 7.5 mg of dexamethasone is recommended with first administration of initial dose of REGN1979 and first administration of the subsequent higher dose (dose step). If the patient tolerates infusions without any signs or symptoms of infusion-related reaction or cytokine release syndrome (CRS), the investigator may lower or eliminate the dose of dexamethasone premedication administered prior to subsequent infusions, as needed based on clinical judgment. Premedication with anti-histamines and/or acetaminophen may also be considered. At doses lower than 300 mcg of REGN1979, empiric premedication with anti-histamines, acetaminophen and/or corticosteroids prior to study drug infusion is not recommended unless the patient has experienced infusion-related reactions or grade 2 or greater CRS with a previous infusion of REGN1979.

It is recommended that patients with ALL who are at high risk for cytokine release syndrome (CRS) and/or TLS (defined by 50% lymphoblasts in bone marrow; lactate dehydrogenase [LDH] ≧500 U/L; or extramedullary involvement) receive a dexamethasone prophase. Dexamethasone prophase should be 10 mg/m2 every day (QD) for a minimum of 3 days and a maximum of 5 days. The dexamethasone prophase must be discontinued ≧72 hours prior to initiation of study drug(s).

At the time of relapse or progression, patients may be considered for retreatment. Patients with a sub-optimal response may also be considered for retreatment with a higher dose of the treatment the patient is already receiving. All decisions for retreatment will be made after discussion between the treating investigator and the sponsor. Retreatment will be at the highest DL that has been deemed safe and tolerable at the time of relapse or progression. Prior to retreatment, patients will be required to re-sign informed consent and meet eligibility criteria for re-treatment.

### Study Endpoints

Time Frame is Baseline to Week 72 (End of study). The primary endpoint is safety (specifically, adverse events [AEs], DLTs, safety laboratory data, and clinical findings). The secondary endpoints are: (i) PK of REGN1979; (ii) Immunogenicity of REGN1979 antibody; (iii) Antitumor activity: (a) Overall response rate as per applicable response criteria for the indication; (b) Duration of response, and progression-free survival at 6 and 12 months; (c) minimal residue disease (MRD) assessment for patients with bone marrow involvement at baseline; and (iv) Pharmacodynamic measures including cytokine profiling, peripheral blood B-cell and T-cell subsets and immune phenotyping, analysis of PD-1 occupancy of circulating T-cells, changes in gene expression in peripheral blood, and serum immunoglobulin.

Percentage change from baseline in the size of target tumor is also noted and summarized.

### Procedures and Assessments

Screening procedures to be performed include cardiac ejection fraction, and brain MRI. Safety procedures include medical history, physical examination, vital signs, electrocardiogram (ECG), coagulation, assessment of B symptoms and evaluation of performance status, clinical laboratory tests, AEs, and concomitant medications.

Efficacy procedures to be performed for tumor assessments include CT or MRI scans, 18F-fluorodeoxyglucose-positron emission tomography (FDG-PET) scans, bone marrow aspirate and biopsies (BMA/Bx), lumbar puncture, lymph node and/or tumor biopsies.

Patients with ALL are assessed according to the NCCN Guidelines 2014.

Assessment for presence of MRD in bone marrow samples is performed centrally by polymerase chain reaction (PCR). Determination of MRD response is performed per Brüggemann et al (Leukemia 2010, 24:521-35) in patients with ALL.

Blood samples for PK and anti-drug antibody (ADA) assessment is collected. Biomarkers samples are collected to monitor for changes in cytokine production, serum levels of pro-inflammatory cytokines, and changes in lymphocyte subsets and activation status. In addition, these samples permit tumor or somatic genetic analyses for variations that impact the clinical course of underlying disease or modulate treatment side effects.

### Safety

An adverse event (AE) any untoward medical occurrence in a patient administered a study drug which may or may not have a causal relationship with the study drug. Therefore, an AE is any unfavorable and unintended sign (including abnormal laboratory finding), symptom, or disease which is temporally associated with the use of a study drug, whether or not considered related to the study drug. An AE also includes any worsening (i.e., any clinically significant change in frequency and/or intensity) of a preexisting condition that is temporally associated with the use of the study drug. Progression of underlying malignancy will not be considered an AE if it is clearly consistent with the typical progression pattern of the underlying cancer (including time course, affected organs, etc.). Clinical symptoms of progression may be reported as AEs if the symptom cannot be determined as exclusively due to the progression of the underlying malignancy, or does not fit the expected pattern of progression for the disease under study. A serious AE (SAE) is any untoward medical occurrence that at any dose results in death, is life-threatening, requires hospitalization, results in persistent or significant disability, and/or is an important medical event.

Patients are monitored for vital signs, general safety, cytokine release syndrome, B-cell depletion, CNS toxicity and for immune-mediated AEs.

### Statistical Plan

Dose escalation cohorts: The study design is based on a traditional 3+3 design with 3 to 6 patients per DL.

Expansion cohorts: The sample size of 20 patients for each expansion cohort is determined based on the clinical consideration to further explore the safety of the Recommended Phase II Dose (RP2D) in the expansion cohorts. The sample size of 20 patients also provides a preliminary evaluation on tumor response.

All AEs reported in this study are coded using the currently available version of the Medical Dictionary for Regulatory Activities (MedDRA®). Coding is to lowest level terms. The verbatim text, the preferred term (PT), and the primary system organ class (SOC) is listed.

Summaries of all treatment-emergent adverse events (TEAEs) by treatment arm include: (i) the number (n) and percentage (%) of patients with at least 1 TEAE by SOC and PT; (ii) TEAEs by severity, presented by SOC and PT; and (iii) TEAEs by relationship to treatment (related, not related), presented by SOC and PT. Deaths and other serious adverse events (SAEs) are listed and summarized by treatment arm. Treatment-emergent adverse events leading to permanent treatment discontinuation are listed and summarized by treatment arm.

### Efficacy Analyses

Objective tumor response, determined by disease-relevant criteria, is summarized. The duration of response and progression-free survival at 6 and 12 months is listed and summarized by the Kaplan-Meier estimator, if needed. Minimal residue disease status is listed and summarized. Progression-free survival is listed and summarized. The percentage change from baseline in the size of the target tumor is also summarized.

### Results

It is expected that the REGN1979 antibody is safe and well-tolerated by patients. It is expected that patients with ALL administered REGN1979 will show tumor growth inhibition and/or remission.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

What is claimed is:

1. A method of treating or inhibiting the growth of leukemic tumor cells in a subject suffering from acute lymphoblastic leukemia, comprising administering to a subject in need thereof a therapeutically effective amount a bispecific antibody comprising a first antigen-binding arm that specifically binds CD20 and a second antigen-binding arm that specifically binds CD3.

2. The method of claim 1, wherein the therapeutically effective amount comprises between 0.1-10 mg/kg of the subject's body weight.

3. The method of claim 2, wherein the therapeutically effective amount comprises 4 mg/kg of the subject's body weight.

4. The method of claim 1, wherein the subject is administered one or more doses of a therapeutically effective amount of the bispecific antibody.

5. The method of claim 4, wherein each dose of the bispecific antibody comprises between 0.1-10 mg/kg of the subject's body weight.

6. The method of claim 4, wherein each dose of the bispecific antibody comprises 4 mg/kg of the subject's body weight.

7. The method of claim 4, wherein each dose of the bispecific antibody comprises between 10-5000 micrograms.

8. The method of claim 4, wherein each dose of the bispecific antibody is administered 0.5-12 weeks after the immediately preceding dose.

9. The method of claim 4, wherein each dose is split into two or more fractions for administration within a dosing period.

10. The method of claim 9, wherein the dose is split into from two to five fractions.

11. The method of claim 1, wherein the bispecific antibody is administered intravenously, subcutaneously, or intraperitoneally.

12. The method of claim 1, wherein the subject is resistant or inadequately responsive to, or relapsed after prior therapy.

13. The method of claim 1, wherein the treatment produces a therapeutic effect selected from the group consisting of delay in reduction in leukemic cell number, increase in survival, partial response, and complete response.

14. The method of claim 13, wherein the therapeutic effect is an increase in survival as compared to an untreated subject.

15. The method of claim 1, wherein the leukemic cell number is reduced by at least 50% as compared to an untreated subject.

16. The method of claim 1 further comprising administering to the subject a second therapeutic agent or therapy, wherein the second therapeutic agent or therapy is selected from the group consisting of radiation, surgery, a chemotherapeutic agent, a cancer vaccine, a PD-1 inhibitor, a PD-L1 inhibitor, a LAG-3 inhibitor, a CTLA-4 inhibitor, a TIM3 inhibitor, a BTLA inhibitor, a TIGIT inhibitor, a CD47 inhibitor, an indoleamine-2,3-dioxygenase (IDO) inhibitor, a vascular endothelial growth factor (VEGF) antagonist, an angiopoietin-2 (Ang2) inhibitor, a transforming growth factor beta (TGFβ) inhibitor, an epidermal growth factor receptor (EGFR) inhibitor, an antibody to a tumor-specific antigen, Bacillus Calmette-Guerin vaccine, granulocyte-macrophage colony-stimulating factor, a cytotoxin, an interleukin 6 receptor (IL-6R) inhibitor, an interleukin 4 receptor (IL-4R) inhibitor, an IL-10 inhibitor, IL-2, IL-7, IL-21, IL-15, an antibody-drug conjugate, an anti-inflammatory drug, and a dietary supplement.

17. The method of claim 1, wherein the first antigen-binding arm of the bispecific antibody comprises three heavy chain CDRs (A-HCDR1, A-HCDR2 and A-HCDR3) of a heavy chain variable region (A-HCVR) comprising the amino acid sequence of SEQ ID NO: 1 and three light chain CDRs (LCDR1, LCDR2 and LCDR3) of a light chain variable region (LCVR) comprising the amino acid sequence of SEQ ID NO: 2.

18. The method of claim 17, wherein A-HCDR1 comprises the amino acid sequence of SEQ ID NO: 4; A-HCDR2 comprises the amino acid sequence of SEQ ID NO: 5; A-HCDR3 comprises the amino acid sequence of SEQ ID NO: 6; LCDR1 comprises the amino acid sequence of SEQ ID NO: 7; LCDR2 comprises the amino acid sequence of SEQ ID NO: 8; and LCDR3 comprises the amino acid sequence of SEQ ID NO: 9.

19. The method of claim 18, wherein the A-HCVR comprises the amino acid sequence of SEQ ID NO: 1 and the LCVR comprises the amino acid sequence of SEQ ID NO: 2.

20. The method of claim 1, wherein the second antigen-binding arm of the bispecific antibody comprises three heavy chain CDRs (B-HCDR1, B-HCDR2 and B-HCDR3) of a heavy chain variable region (B-HCVR) comprising the amino acid sequence of SEQ ID NO: 3 and three light chain CDRs (LCDR1, LCDR2 and LCDR3) of a light chain variable region (LCVR) comprising the amino acid sequence of SEQ ID NO: 2.

21. The method of claim 20, wherein B-HCDR1 comprises the amino acid sequence of SEQ ID NO: 10; B-HCDR2 comprises the amino acid sequence of SEQ ID NO: 11; B-HCDR3 comprises the amino acid sequence of SEQ ID NO: 12; LCDR1 comprises the amino acid sequence of SEQ ID NO: 7; LCDR2 comprises the amino acid sequence of SEQ ID NO: 8; and LCDR3 comprises the amino acid sequence of SEQ ID NO: 9.

22. The method of claim 21, wherein the B-HCVR comprises the amino acid sequence of SEQ ID NO: 3 and the LCVR comprises the amino acid sequence of SEQ ID NO: 2.

23. The method of claim 22, wherein the bispecific antibody is REGN1979.

24. The method of claim 1, wherein the subject has CD20 expression on ≧0% of leukemic lymphoblasts, as determined by flow cytometry.

25. The method of claim 24, wherein the subject has CD20 expression on ≧15% of leukemic lymphoblasts, as determined by flow cytometry.

26. The method of claim 25, wherein the subject has CD20 expression on ≧20% of leukemic lymphoblasts, as determined by flow cytometry.

27. The method of claim 1, wherein the bispecific antibody further comprises a chimeric Fc domain tethered to each of the first and second antigen-binding domains.

28. The method of claim 27, wherein the chimeric Fc domain comprises a chimeric hinge.
